(19) United States
(12) Patent Application Publication
(10) Pub. No.: US 2010/0114593 A1
(43) Pub. Date: May 6, 2010

(54) MEANS AND METHOD OF OBTAINING AND PROCESSING DATA FOR USE IN MEDICAL OR HEALTH ASSESSMENT

(75) Inventors: Oded Sarel, Even Yehuda (IL); Moty Bahar, Kfar Azar (IL)

Correspondence Address: The Law Office of Michael E. Kondoudis, 888 16th Street, N.W., Suite 800, Washington, DC, 20006, US

(73) Assignee: MEDIRISK SOLUTIONS LTD., Tel Aviv (IL)

(21) Appl. No.: 12/377,213

(22) PCT Filed: Jun. 25, 2007
(86) PCT No.: PCT/IL2007/000768

§ 371 Date: Feb. 11, 2009

(30) Foreign Application Priority Data
Jun. 25, 2006 (IL) 176539

(51) Int. Cl. G06Q 40/00; G06Q 50/00; G06F 17/30

(52) U.S. Cl. 705/2; 705/4; 707/801; 707/E17.011

(57) ABSTRACT

Present invention discloses a system and a method of obtaining and processing data for use in insurance underwriting which comprise a set of influencing factors, provide an application questionnaire including a series of application questions relating to a plurality of influencing factors and a plurality of possible application responses to each application question, assign risk-scores to at least a portion of said application responses, present said application questionnaire to an applicant, collect application responses to at least a portion of the application questions, compile optionally at least one expert questionnaire including all questions left unanswered by the applicant, present optionally said expert questionnaires to at least one expert, and collect responses to the expert questionnaires and determine a total response for the applicant.

### FIELD OF THE INVENTION

The present invention generally relates to a means and method of obtaining and processing data for use in medical or health assessment.

### BACKGROUND OF THE INVENTION

The role of the underwriter has remained virtually untouched since insurance first began. The applicant answers a number of questions on the proposal form, which arrives at the insurer's new business department where it is determined whether the case can be accepted on normal terms or if more information is required to help the underwriter in assessing the risk.

The underwriting process is commonly understood in three phases:
1. initial underwriting;
2. first stage of full underwriting; and
3. second stage of full underwriting.

All applications go through the initial underwriting process, many cases are accepted at this stage, such stages are known in the field as ‘clean’ cases. Clean cases do not usually proceed to full underwriting.

Cases that were not accepted in the initial underwriting process proceed to the first stage of full underwriting. Here they undergo a more detailed assessment and an underwriter decides if the case can be accepted, or whether additional information or evidence, for example from the family doctor of the applicant, is required before a final decision can be made.

The second stage of full underwriting is completed after further evidence has been received. At this point, the case may be accepted with certain exclusions, a loaded premium, or cover could be refused.

Although an underwriter should not rely on his/her judgement alone to make decisions, there are no formal training programmes to prepare an underwriter for this specialist role. Reinsurance companies often help by providing manuals and holding seminars to arm underwriters with as much knowledge as possible. In a number of instances, these guides have been developed into electronic form in order to make them easier to use and to start automating some aspects of the process. Nevertheless the underwriter relies largely upon his own experience and as are result, a subjective element is introduced into the underwriting process.

To assist the underwriter, a life assurance company will utilise the services of their Chief Medical Officer, a fully qualified doctor, to provide medical expertise in the more complex cases. However even a qualified does not have a detailed knowledge of all aspects of medicine and diseases.

For the typical non-clean application, the underwriting process can be quite time consuming. On average such an application takes between 3 to 6 weeks to be processed but can take much longer, for example where the family doctor does not return their report promptly. Thus once a proposal has been submitted, long delays and lack of information are translated into negative customer perceptions at just the point where building trust and relationship is so important to both parties.

This time delay presents economic strain upon the insurer in two ways. Firstly, the labour costs of the underwriter are high, as it is typically the most experienced and therefore most expensive personnel who process the most difficult and therefore the most time consuming cases. In addition the time delay has a negative influence on sales themselves, the longer it takes to process the application, the greater the probability that the applicant will withdraw.

Nevertheless, the more information that the underwriter can access during the application the smaller the risk will be to insurer. To this end it is necessary to develop methods by which as much accurate information can be gathered as quickly as possible. Over the last twenty years traditional sources of information have been becoming progressively less reliable. This is for a variety of reasons; firstly legislation has limited the freedom with which a family doctor can divulge medical information where the doctor feels it is not in the interest of the patient. In addition the rate of medical progress is such that it is impossible for a Chief Medical Officer to keep abreast of all the latest developments.

Automated underwriting systems have been developed to improve the efficiency of the process. Most of these systems comprise of a data gathering phase and a processing phase. The data gathering phase might make use tele-interviewing, or the gathering information directly from the applicant particularly over the phone. Various models exist for tele-interviewing but in general it is an efficient way for less expensive personnel to gather information for processing.

So, for example, WO patent application 03058380 describes a system to structure and summarize the information obtained from doctors' report by capturing relevant variables that characterize a given medical impairment, which could allow an automated reasoning system to determine the degree of severity of such impairment and to thus estimate the underlying insurance risk. This invention is specifically aims to standardize the medical report. More generally, US patent application 2005144047 describes a system comprising a plurality of browsers each of which serves as a station at which information can be entered and communicated to a dispatcher and parses the information to be used in the selection of an insurance process.

The processing phase can also be automated by means of rules. Presently rules take the form of if-when conditions with each condition providing a unique rule. Even systems such as that described in WO patent application 2003065268 which provide for the possibility of fuzzy logic, allowing intermediate values between absolute true and false, do so by means of creating two if-then conditions each of which determines one extreme of the range of allowed values.

The outcome of these rules can determine for example if an applicant should be rejected, accepted, or conditionally accepted either with additional premium or with certain exclusions. However because each rule needs to be entered individually the initial set up of the system is very time consuming and once set up the system is not easily updated. As a result it is not cost effective to create rules describing less common cases and the rules cannot rapidly respond to advances in medicine. Therefore current automated systems provide only limited automation and usually only of the initial underwriting phases.

What is needed is an adaptable software system for the medical underwriting process, producing an accurate, objective, reliable and rapid underwriting decision for any given insurance product based on the specific insurer underwriting philosophy.

Thus there remains a long felt need for the present invention relating to a means and method of obtaining and processing data for use in medical or health assessment.

### SUMMARY OF THE INVENTION

It is thus one object of the present invention to teach a method of obtaining and processing data for use in insurance underwriting comprising; compiling a set of influencing factors; providing an application questionnaire including a series of application questions relating to a plurality of influencing factors; providing a plurality of possible application responses to each application question; assigning risk-scores to each application response; presenting said application questionnaire to an applicant; collecting application responses to at least a portion of the application questions; compiling at least one expert questionnaire including all questions left unanswered by the applicant; presenting said expert questionnaires to at least one expert; collecting responses to the expert questionnaires; and determining a total risk-score for the applicant.

It is another object of the present invention to teach a method additionally providing a computerized application questionnaire wherein only those application questions which are relevant to the applicant are presented to the respondent.

It is another object of the present invention to teach a method additionally providing medical insurance underwriting by producing an expert questionnaire, for a medical expert, including the specific application questions for which application responses are necessary and obtaining the applicants consent for the medical expert to reveal this information.

It is another object of the present invention to teach a method additionally including a plurality of application questions with a plurality of selectable application responses.

It is another object of the present invention to teach a method additionally including a plurality of application questions with a plurality of open application responses.

It is another object of the present invention to teach a method additionally comprising an initial steps of (a′) obtaining a database arranged as a multiple dimensional array of relevant records; wherein various diseases are listed in one or more iₙ records; and various symptoms are listed in one or more other jₙ records; and (b′) correlating a single or multiple correlations between those iₙjₙ conjugated records, in a manner that more weight is attributed to factors (e.g., diseases) characterized by more other records (e.g., symptoms).

It is another object of the present invention to teach a method especially useful for determining the total risk-score of said applicant, additionally comprising assigning a plurality of changeable risk-score values for one or more parameters selected from a group consisting of said iₙ records, jₙ records, and iₙjₙ conjugated records.

It is another object of the present invention to present a means of obtaining and processing data for use in insurance underwriting comprising; a set of influencing factors; an application questionnaire including a series of application questions relating to a plurality of influencing factors; a plurality of possible application responses to each application question; risk-scores assigned to each application response; a means of presenting said application questionnaire to an applicant; a means of collecting application responses to at least a portion of the application questions; at least one expert questionnaire including all questions left unanswered by the applicant; a means of presenting said expert questionnaires to at least one expert; a means of collecting responses to the expert questionnaires; and a means of determining a total risk-score for the applicant.

It is another object of the present invention to present a means additionally comprising a computerized application questionnaire wherein only those application questions which are relevant to the applicant are presented to the respondent.

It is another object of the present invention to present a means for use in medical insurance underwriting additionally comprising an expert questionnaire, for a medical expert, including the specific application questions for which application responses are necessary and obtaining the applicants consent for the medical expert to reveal this information.

It is another object of the present invention to present a means additionally comprising a plurality of application questions with a plurality of selectable application responses.

It is another object of the present invention to present a means additionally comprising a plurality of application questions with a plurality of open application responses.

It is another object of the present invention to present a means additionally comprising a database arranged as a multiple dimensional array of relevant records; wherein various diseases are listed in one or more iₙ records; and various symptoms are listed in one or more other jₙ records; and a single or multiple correlations correlated between those iₙjₙ conjugated records, in a manner that more weight is attributed to factors (e.g., diseases) characterized by more other records (e.g., symptoms).

It is another object of the present invention to present a means especially useful for the determination of the total risk-score of said applicant, additionally a plurality of changeable risk-score values that are assigned for one or more parameters selected from a group consisting of said iₙ records, jₙ records, and iₙjₙ conjugated records.

### BRIEF DESCRIPTION OF THE FIGURES

The objects and advantages of various embodiments of the invention will become apparent from the following description when read in conjunction with the accompanying drawings wherein;

FIG. 1 presents the flow diagram associated with the algorithm followed when obtaining and processing data, according to one embodiment of the current invention;

FIG. 2 presents an alternative flow diagram associated with the algorithm followed when obtaining and processing data of a numerical type, according to another embodiment of the current invention;

FIG. 3 presents an example of a screen-shot of a first level question as displayed in a web-browser application questionnaire, according to another embodiment of the current invention;

FIG. 4 presents an example of a screen-shot of a second level question as displayed in a web-browser application questionnaire, according to another embodiment of the current invention;

FIG. 5 presents an example of a screen-shot of a third level question as displayed in a web-browser application questionnaire, according to another embodiment of the current invention;

FIG. 6 presents an example of a screen-shot of a fourth level question as displayed in a web-browser application questionnaire, according to another embodiment of the current invention;

FIG. 7 presents an example of a screen-shot of a first level question as displayed in a web-browser application questionnaire, in which both options are selected, according to another embodiment of the current invention;

FIG. 8 presents an example of a screen-shot of a second level question as displayed in a web-browser application questionnaire, according to another embodiment of the current invention;

FIG. 9 presents an example of a screen-shot of the response confirmation screen, from a web-browser application questionnaire, according to another embodiment of the current invention;

FIG. 10 presents an example of a screen-shot of the underwriting decision screen of a web-browser application questionnaire, according to another embodiment of the current invention; and

FIG. 11 presents an example of a screen-shot of a case summary as displayed in a web-browser application questionnaire, according to another embodiment of the current invention.

### DETAILED DESCRIPTION OF THE EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method of obtaining and processing data for use in medical or health assessment.

The term ‘plurality’ refers hereinafter to any number greater than or equal to one.

The terms ‘about’ or ‘approximately’ in the context of a numerical value refers hereinafter to any value in a range from 20% below to 20% above the stated value.

The term ‘ease’ refers hereinafter to any subject undergoing assessment. For example a case could be, in a non-limiting manner, an individual applying for life assurance, a shipping company applying for insurance cover for a cargo, a patient undergoing diagnosis, a subject of health assessment or any other subject being assessed.

The term ‘underwriting’ refers hereinafter to the process whereby an insurer or reinsurer reviews applications submitted for insurance or reinsurance coverage and determines whether it will provide all or part of the coverage being requested and at what premium.

The term ‘insurance’ refers hereinafter to a plan whereby a party will pay premiums to a second party, who in return, will reimburse the first party in case of loss.

The term ‘reinsurance’ refers hereinafter to an insurance plan protecting wherein one insurer will pay premiums to a second insurer, who in return, will at least partially reimburse the first insurer in case of a claim being made against them.

The term ‘insurer’ refers hereinafter to a party providing insurance cover.

The term ‘influencing factor’ refers hereinafter to any agent which causally affects an outcome. In particular this refers to a condition which may increase or decrease the probability of a particular outcome.

The term ‘risk-score value’ refers hereinafter to a value assigned to a given combination of factors and which reflects the degree to which said combination of factors influences the probability of an outcome.

The term ‘total risk-score’ refers hereinafter to the overall risk-score reflecting the influence of all known influencing factors of a particular case. This total risk-score can be calculated, for example, by summing or averaging all the risk-scores assigned to the combinations of all pair of influencing factors in a particular case.

The term ‘application question’ refers hereinafter to a question asked to an insurance applicant in order to gain information pertaining to the risk associated with their insurance cover.

The term ‘application response’ refers hereinafter to information obtained in response to an application question.

The term ‘tele-interviewing’ refers hereinafter to the remote gathering of data by either manual or automatic means. It is noted that said gathering of data may be at the point of sale and may be effected by means of telephone, interne, computer station or any other means or data gathering.

The term ‘premium’ refers hereinafter to regular periodic payments, that a policyholder makes to own an insurance policy.

The term ‘exclusions’ refers hereinafter to specific risks within an insurance policy which the insurer specifies as not covered in a particular policy.

The term ‘extent of cover’ refers hereinafter to the existence or otherwise of exclusions in a policy.

The term ‘insurance type’ refers hereinafter to the nature of the risk covered by an insurance policy.

The term ‘manual override’ refers hereinafter to a means for an underwriter to determine the premium and extent of cover associated to a given insurance policy independently of any data held by a system.

It is according to one embodiment of the present invention to teach a method of obtaining and processing data for use in insurance underwriting comprising; compiling a set of influencing factors; providing an application questionnaire including a series of application questions relating to a plurality of influencing factors; providing a plurality of possible application responses to each application question; assigning risk-scores to each application response; presenting said application questionnaire to an applicant; collecting application responses to at least a portion of the application questions; compiling at least one expert questionnaire including all questions left unanswered by the applicant; presenting said expert questionnaires to at least one expert; collecting responses to the expert questionnaires; and determining a total risk-score for the applicant.

It is according to another embodiment of the present invention to teach a method additionally providing a computerized application questionnaire wherein only those application questions which are relevant to the applicant are presented to the respondent.

It is according to another embodiment of the present invention to teach a method additionally providing medical insurance underwriting by producing an expert questionnaire, for a medical expert, including the specific application questions for which application responses are necessary and obtaining the applicants consent for the medical expert to reveal this information.

It is according to another embodiment of the present invention to teach a method additionally including a plurality of application questions with a plurality of selectable application responses.

It is according to another embodiment of the present invention to teach a method additionally including a plurality of application questions with a plurality of open application responses.

It is according to another embodiment of the present invention to present a means of obtaining and processing data for use in insurance underwriting comprising; a set of influencing factors; an application questionnaire including a series of application questions relating to a plurality of influencing factors; a plurality of possible application responses to each application question; risk-scores assigned to each application response; a means of presenting said application questionnaire to an applicant; a means of collecting application responses to at least a portion of the application questions; at least one expert questionnaire including all questions left unanswered by the applicant; a means of presenting said expert questionnaires to at least one expert; a means of collecting responses to the expert questionnaires; and a means of determining a total risk-score for the applicant.

It is according to another embodiment of the present invention to present a means additionally comprising a computerized application questionnaire wherein only those application questions which are relevant to the applicant are presented to the respondent.

It is according to another embodiment of the present invention to present a means for use in medical insurance underwriting additionally comprising an expert questionnaire, for a medical expert, including the specific application questions for which application responses are necessary and obtaining the applicants consent for the medical expert to reveal this information.

It is according to another embodiment of the present invention to present a means additionally comprising a plurality of application questions with a plurality of selectable application responses.

It is according to another embodiment of the present invention to present a means additionally comprising a plurality of application questions with a plurality of open application responses.

It is noted with regard to the means and method herein described that;
1. the assessment of both individuals and groups of individuals is enabled,
2. an additional engine is optionally included providing an interface between a database or plurality of databases containing additional data such as an applicants application history, claim history or any other relevant data,
3. a means is provided for the storage of all data obtained such that where a user does not complete the questionnaire the current status of responses is saved and a user may reaccess said in order to complete the questionnaire,
4. any question may illicit at any stage at least one of four possible outputs including
   a. grading output,
   b. formula output,
   c. acceptance including exceptions, or
   d. absolute rejection.
5. the data used for the assessment may be obtained from a plurality of sources,
6. the questionnaire can be provided in a form which allows any professional or non-professional user to input the relevant data,
7. assessment can be provided for a plurality of purposes such as health or medical assessment, insurance underwriting, visa applications, individual profiling for example for military assessment, assessment of effectively of products such as medicines, assessment of population groups, assessment of severity of illness, longevity,
8. assessment for a particular case or group of cases is based upon the data gathered for that case in a manner such that the assessment grading is updated as each additional datum is obtained, and
9. the accuracy of the assessment can increase as the quantity of data increases.

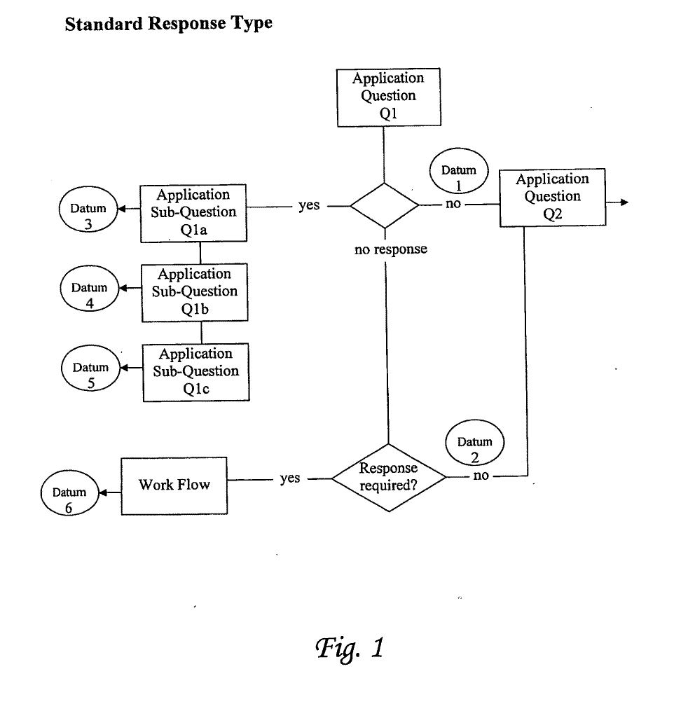

Reference is now made to FIG. 1 which presents the flow diagram associated with the algorithm followed when obtaining and processing data, according to one embodiment of the current invention. A question is asked of the respondent that can be answered by either “yes”, “no” or no response might be given, for example in the case of the respondent not knowing the correct response. In the event of a “yes” response, lower level questions are presented to the respondent, which have similar response structures. When questions of the lowest order are answered a datum output is returned to be stored in a database for analysis. In the event of a “no” response, a datum output is returned and the respondent is presented with the next question. In the event of no response being given, there are two possibilities, when no response is required for the question the respondent is presented with the next question and a datum output may be returned to the database. If the response is a required factor, further clarification is necessary so a work flow is put into action. This workflow might include a restatement of the question with a different wording or, more typically, the question can be included in a questionnaire sent to a doctor for clarification.

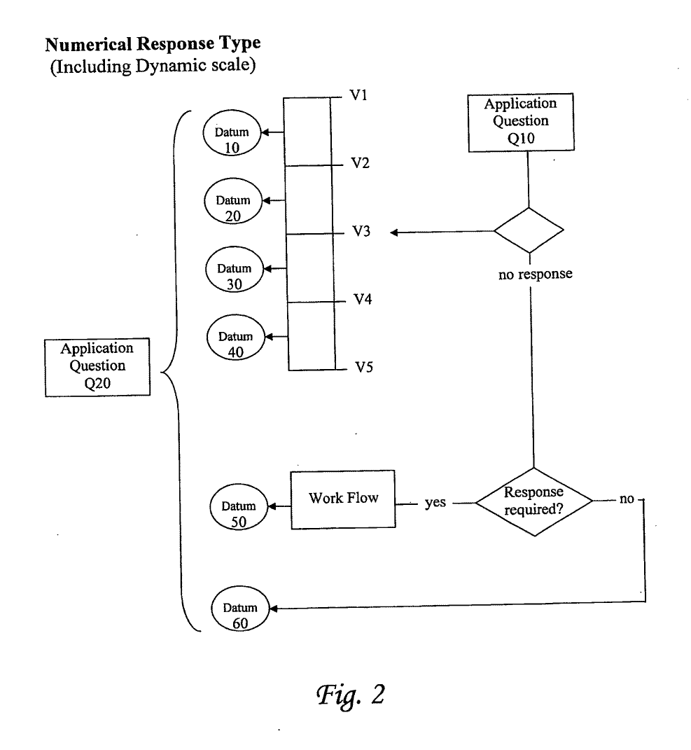

Reference is now made to FIG. 2 which presents an alternative flow diagram associated with the algorithm followed when obtaining and processing data of a numerical type, according to another embodiment of the current invention. A question is asked of the respondent that can be answered by either a numerical response or no response might be given, for example in the case of the respondent not knowing the correct response. Numerical responses are given by dividing a scale into sub-ranges, here V1 to V2 represents the first range, V2 to V3 the second range and so on. Each sub-range, if selected returns a datum output, so for example datum 10 is returned by the sub-range between V1 to V2. It should be noted that the divisions of this scale is variable and can varied to suit the application. As with the standard case presented in FIG. 1, in the event of no response being given, there are two possibilities, when no response is required for the question the respondent is presented with the next question and a datum output may be returned to the database. If the response is a required factor, further clarification is necessary so a work flow is put into action. This workflow might include a restatement of the question with a different wording or, more typically, the question can be included in a questionnaire sent to a doctor for clarification.

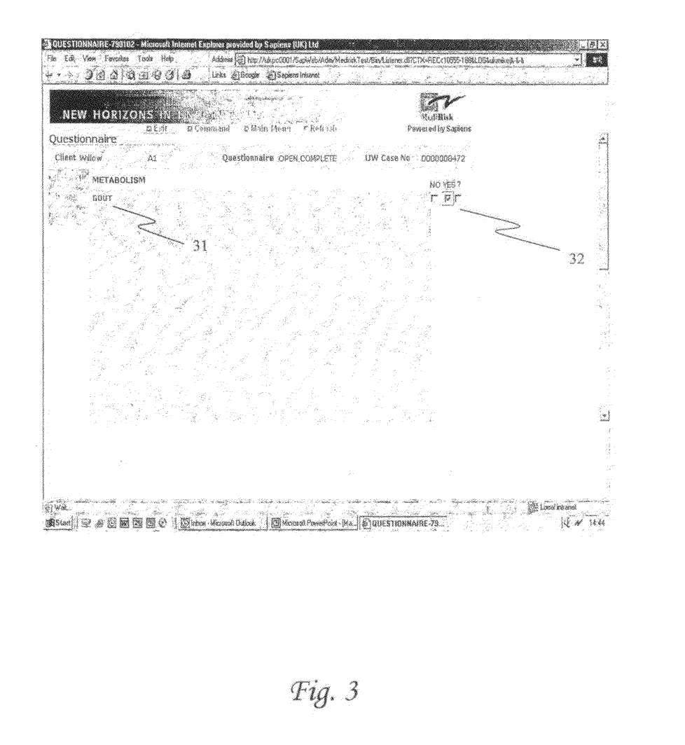

Reference is now made to FIG. 3 which presents an example of a screen-shot of a first level question as displayed in a web-browser application questionnaire, according to another embodiment of the current invention. An applicant is presented with this screen as a part of their application, the question here, 31, is “Do you suffer from Gout?” the applicant is offered three possible responses: No, Yes or Unsure, 32. Here the Yes option has been selected. This leads to a second level question.

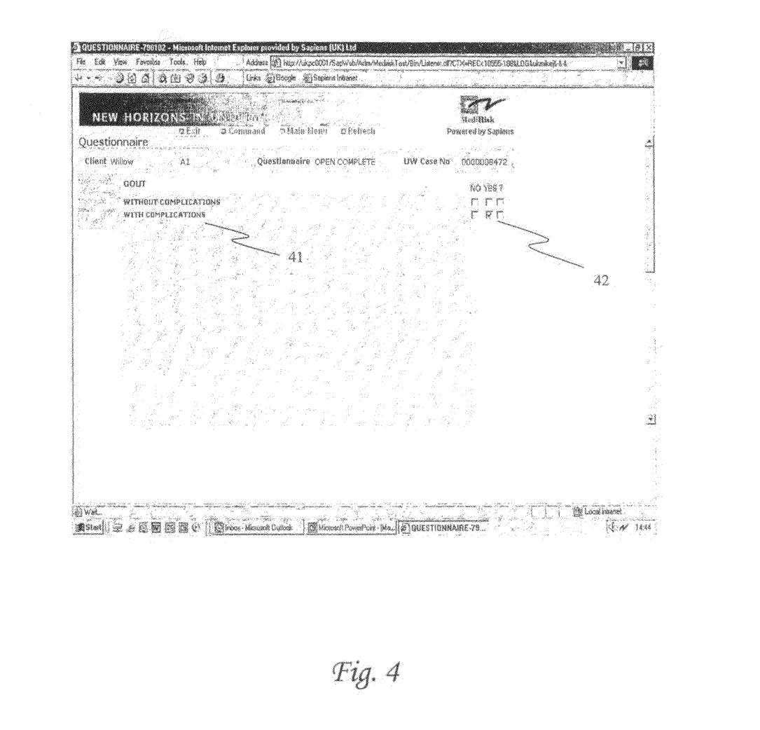

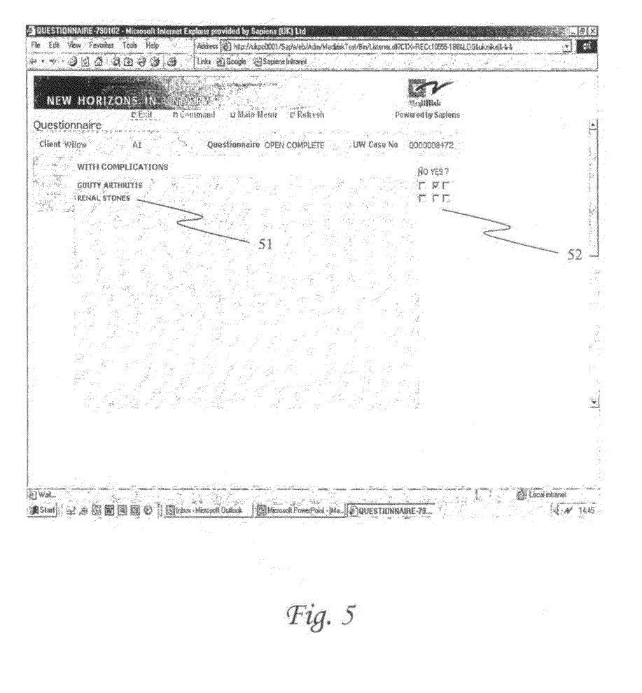

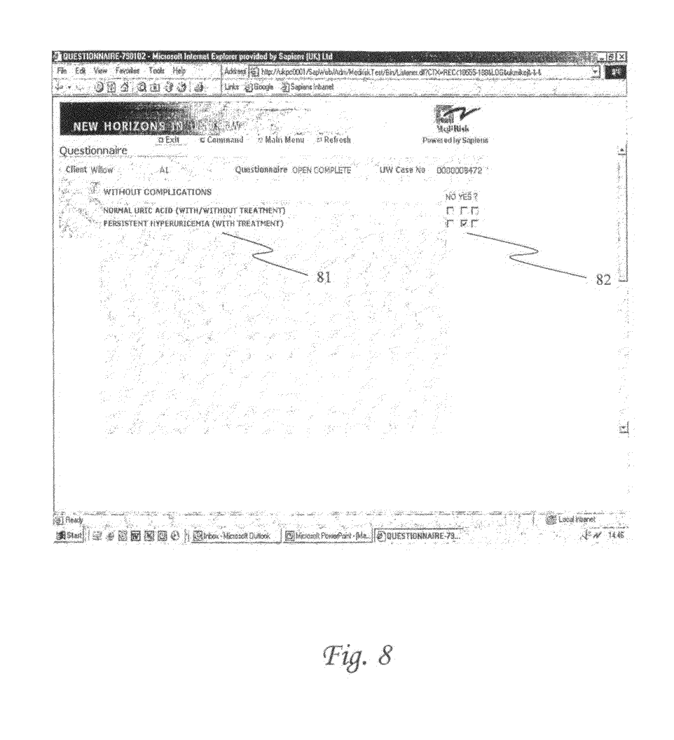

Reference is now made to FIG. 4 which presents an example of a screen-shot of a second level question as displayed in a web-browser application questionnaire, according to another embodiment of the current invention. Here the applicant is asked further details about their gout. On this screen they are asked if their gout is with or without complications, 41. The applicant is offered three possible responses: No, Yes or Unsure, 42, for each question. Each response leads to a different third level question, selection of Yes to “without complications” would lead to the third level question shown in FIG. 8, whereas selection of Yes to “with complications”, as shown leads to the third level question shown in FIG. 5.

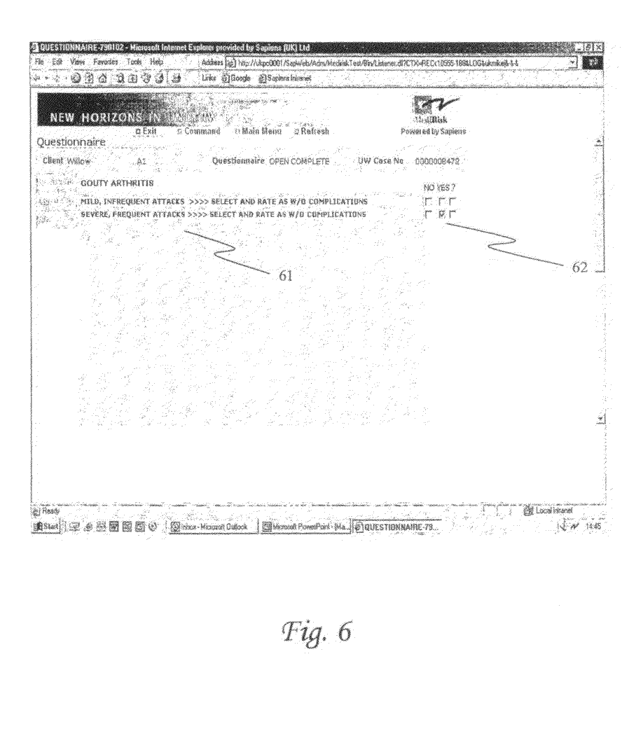

Reference is now made to FIG. 5 which presents an example of a screen-shot of a third level question as displayed in a web-browser application questionnaire, according to another embodiment of the current invention. The same structure repeats as was seen in the higher order to questions. With questions, 51, and the possible responses, 52, being offered for selection. Selection here leads to a fourth level question ascertaining the severity of the disorder as shown in FIG. 6.

Reference is now made to FIG. 6 which presents an example of a screen-shot of a fourth level question as displayed in a web-browser application questionnaire, according to another embodiment of the current invention. Again there is the same structure here as in the higher order to questions. With questions, 61, and the possible responses, 62, being offered for selection. As this is the lowest level question along this thread, the response here sends a datum to a data base where is stored for processing along with data gathered from all other responses. The applicant is now returned to the second level again.

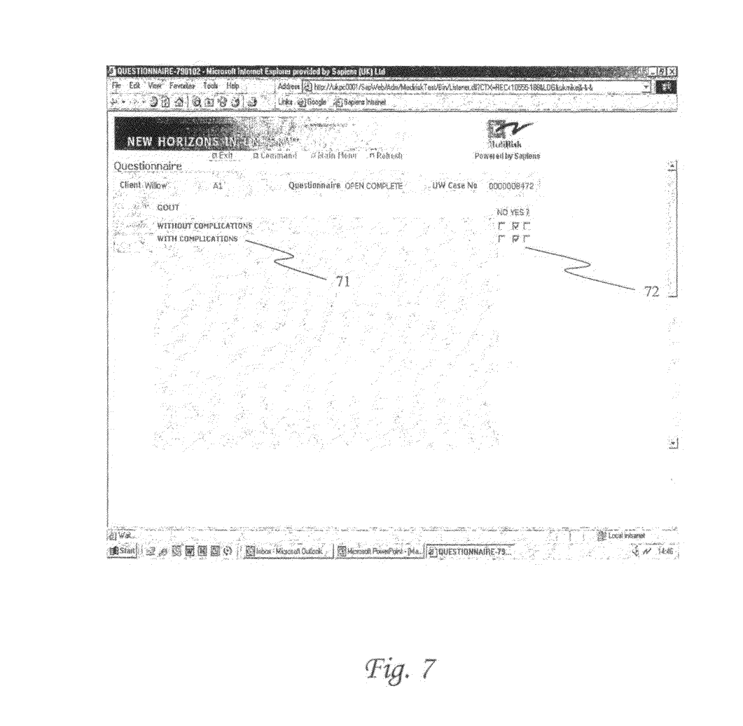

Reference is now made to FIG. 7 which presents an example of a screen-shot of the same second level question as presented in FIG. 4, according to another embodiment of the current invention. In this case however the Yes option has been selected for both the “with complications” and the “without complications”.

Reference is now made to FIG. 8 which presents an example of a screen-shot of a third level question as displayed in a web-browser application questionnaire, according to another embodiment of the current invention. This is the next question along the thread from “gout” to “without complications”.

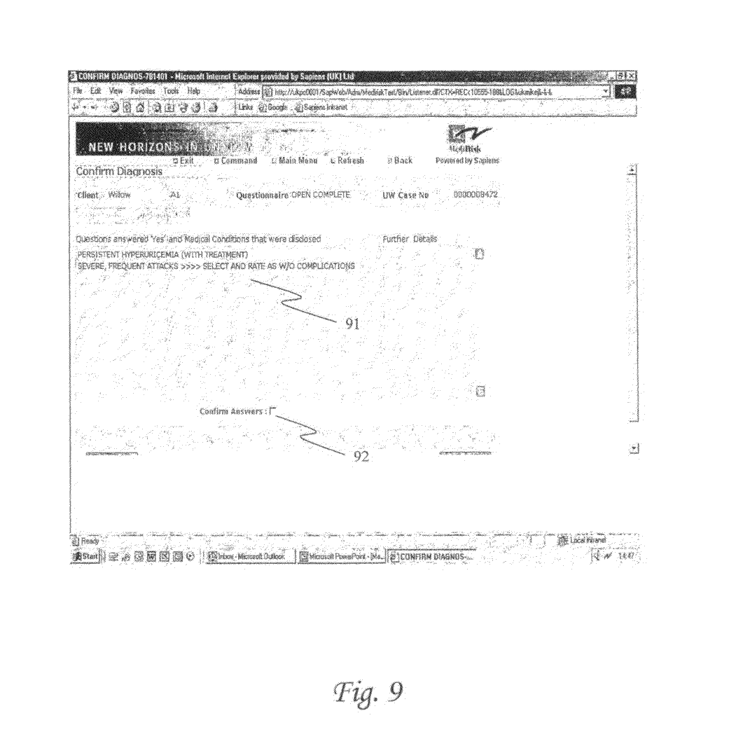

Reference is now made to FIG. 9 which presents an example of a screen-shot of the response confirmation screen, from a web-browser application questionnaire, according to another embodiment of the current invention. The list of responses made is presented in the pane, 91. By ticking the tick-box, 92, the applicant confirms the information.

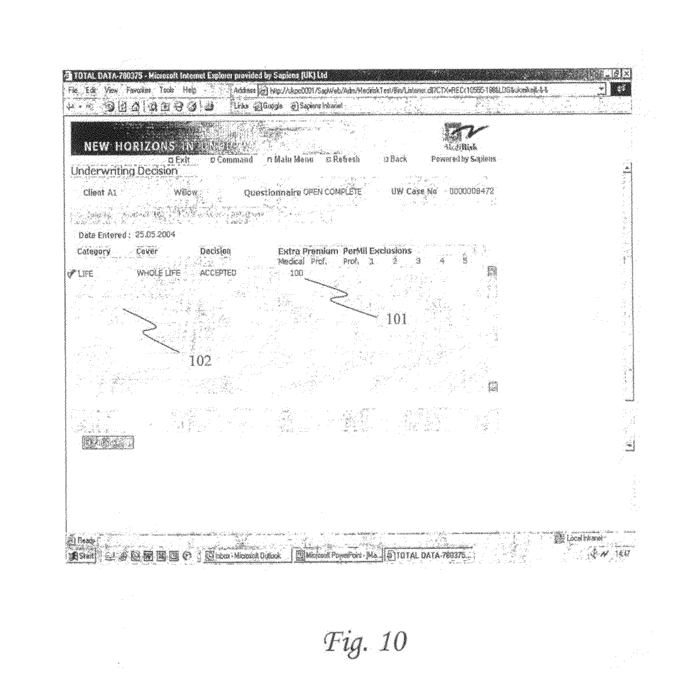

Reference is now made to FIG. 10 which presents an example of a screen-shot of the underwriting decision screen of a web-browser application questionnaire, according to another embodiment of the current invention. In many cases the insurance can be confirmed at the point of application. In this example the information has determined an extra premium of 100 for life assurance cover. Although in this example only life assurance results are displayed, additional results can be presented in this pane, 102, for other insurances such as medical insurance, disability insurance, nursing insurance or other forms of cover, all results are based upon the same data.

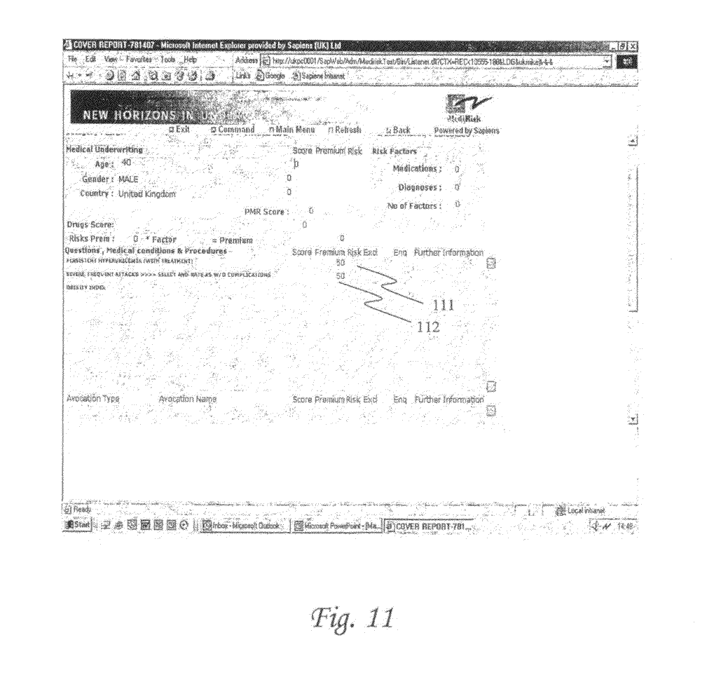

Reference is now made to FIG. 11 which presents an example of a screen-shot of a case summary as displayed in a web-browser application questionnaire, according to another embodiment of the current invention. Here can be seen the break down of the extra premium value of 100. An extra premium value of 50 is due to the hyperuricemia, 111, and a further 50 due to the severity of the gout attacks, 112.

It is also in the scope of the present invention, wherein the aforesaid data for use in insurance underwriting comprises or calculated in function with correlations made between a plurality of parameters, e.g., parameters related with a plurality of applicant's influencing factors, risk-scores etc., wherein the data is provided in a multiple dimensional array or relevant records. Hence, as illustrated in a 2D database for example, various diseases, such as eye diseases, kidney diseases etc are listed in one or more iₙ records; symptoms, such as diabetes, arthritis, anaemia etc are listed in one or more other jₙ records. A single or multiple correlations are made between those iₙjₙ conjugated records, in a manner that e.g., more weight is attributed to factors (e.g., diseases) characterized by more other records (e.g., symptoms). This multi-dimensional array of data enables the insurer to introduce the applicant updated and concise application questioner. It is thus in the scope of the invention wherein a specifically targeted questionnaire and/or check list for filing an application question as defined in any of the above is provided. It is also in the scope of the invention wherein said multiple dimension data comprising changeable risk-score values assigned for one or more parameters of a group consisting inter alia of the aforesaid iₙ records, jₙ records, and iₙjₙ conjugated records determining the total risk-score.

1. A method of obtaining and processing data for use in insurance underwriting;
said method comprising;
a. compiling a set of influencing factors;
b. providing an application questionnaire including a series of application questions relating to a plurality of influencing factors;
c. providing a plurality of possible application responses to each application question;
d. assigning risk-scores to at least a portion of said application responses;
e. presenting said application questionnaire to an applicant;
f. collecting application responses to at least a portion of the application questions;
g. optionally compiling at least one expert questionnaire including all questions left unanswered by the applicant;
h. optionally presenting said expert questionnaires to at least one expert;
i. collecting responses to the expert questionnaires; and,
j. determining a total response for the applicant.

2. The method according to claim 1, additionally providing a computerized application questionnaire wherein only those application questions which are relevant to the applicant are presented to the respondent.

3. The method according to claim 1, additionally providing medical insurance underwriting by producing an expert questionnaire, for a medical expert, including the specific application questions for which application responses are necessary and obtaining the applicants consent for the medical expert to-reveal this information.

4. The method according to claim I, additionally including a plurality of application questions with a plurality of selectable application responses.

5. The method according to claim 1, additionally including a plurality of application questions with a plurality of open application responses.

6. The method according to claim 1, additionally comprising an initial steps of (a′) obtaining a database arranged as a multiple dimensional array of relevant records; wherein various diseases are listed in one or more iₙ records; and
various symptoms are listed in one or more other jₙ records; and (b′) correlating a single or multiple correlations between those iₙjₙ conjugated records, in a manner that more weight is attributed to factors (e.g., diseases) characterized by more other records (e.g., symptoms).

7. The method according to claim 6 useful for determining the total risk-score of said applicant, additionally comprising assigning a plurality of changeable risk-score values for one or more parameters selected from a group consisting of said iₙ records, jₙ records, and iₙjₙ conjugated records.

8. An insurance underwriting system of obtaining and processing data for use in insurance underwriting; said system comprising;
a. a set of influencing factors;
b. an application questionnaire including a series of application questions relating to a plurality of influencing factors;
c. a plurality of possible application responses to each application question;
d. risk-scores assigned to each application response;
e. a means of presenting said application questionnaire to an applicant;
f. a means of collecting application responses to at least a portion of the application questions;
g. at least one expert questionnaire including all questions left unanswered by the applicant;
h. a means of presenting said expert questionnaires to at least one expert;
i. a means of collecting responses to the expert questionnaires; and,
j. a means of determining a total risk-score for the applicant.

9. The means, according to claim 8, additionally comprising a computerized application questionnaire wherein only those application questions which are relevant to the applicant are presented to the respondent.

10. The means, according to claim 8, for use in medical insurance underwriting additionally comprising an expert questionnaire, for a medical expert, including the specific application questions for which application responses are necessary and obtaining the applicants consent for the medical expert to reveal this information.

11. The means, according to claim 8, additionally comprising a plurality of application questions with a plurality of selectable application responses.

12. The means, according to claim 8, additionally comprising a plurality of application questions with a plurality of open application responses.

13. The means according to claim 8, additionally comprising a database arranged as a multiple dimensional array of relevant records; wherein various diseases are listed in one or more iₙ records; and various symptoms are listed in one or more other jₙ records; and a single or multiple correlations correlated between those iₙjₙ conjugated records, in a manner that more weight is attributed to factors (e.g., diseases) characterized by more other records (e.g., symptoms).

14. The means according to claim 13 useful for the determination of the total risk-score of said applicant, additionally a plurality of changeable risk-score values that are assigned for one or more parameters selected from a group consisting of said iₙ records, jₙ records, and iₙjₙ conjugated records.
